(19) United States
(12) Patent Application Publication
(10) Pub. No.: US 2015/0230803 A1
(43) Pub. Date: Aug. 20, 2015

(54) Rapid application tourniquet

(71) Applicants: Richard Hill Schreckengaust, Emerald Isle, NC (US); Paul Hetzel, Annapolis, MD (US)

(72) Inventors: Richard Hill Schreckengaust, Emerald Isle, NC (US); Paul Hetzel, Annapolis, MD (US)

(21) Appl. No.: 14/180,772

(22) Filed: Feb. 14, 2014

(51) Int. Cl. A61B 17/135

(52) U.S. Cl. CPC A61B 17/135

(57) ABSTRACT

A tourniquet system for rapid automatic application of adequate pressure to stem bleeding of an injured limb while minimizing tissue damage due to excessive pressure. Pressure is supplied to the limb by inflation of a tourniquet cuff with compressed gas. The invention also relates to a method of controlling bleeding from a limb using the tourniquet system.

### CROSS-REFERENCES TO RELATED APPLICATIONS

This application claims the benefit of U.S. application Ser. No. 61/766,787 filed Feb. 20, 2013, which is incorporated herein by reference.

### FIELD OF THE INVENTION

The inventive subject matter relates to a tourniquet that is able to be rapidly and easily applied and a method of controlling blood loss from a limb using the tourniquet.

### BACKGROUND OF INVENTION

Minimizing the loss of blood is an important in reducing the risk of organ damage, or death, following injury, especially in traumatic wounds. An objective, after prevention or reduction of blood loss, to minimize the damage to tissue or limbs that might arise resulting from excessive pressure under the tourniquet.

In certain circumstances, such as individuals injured away from the availability of healthcare facilities, application of effective blood loss devices necessitates that a tourniquet be applied rapidly, either by untrained individuals in the area or even potentially by the injured individual.

An emergency tourniquet (Aginsky (U.S. Pat. No. 4,243,039)) utilizes a strap and tensioning device. In this system, after the tourniquet is in place, pressure on the limb is increased by winding the strap around a spool on the tensioning device. Pressure is monitored by a scale combined with a pointer to indicate permissible pull by the tensioning device.

With currently available systems, however, significant discomfort occurs as pressure is applied, making self application difficult. Furthermore, regulating adequate pressure to relieve blood loss while mitigating limb or tissue damage is difficult.

### SUMMARY OF INVENTION

An object of this invention is a pneumatic tourniquet device for control of bleeding of a limb that is easy to apply, even by wearer, rapidly but with adequate pressure to stem bleeding of the injured limb. The device comprises a cuff that is secured to a limb with control bleeding provided by pressure supplied by inflation of the cuff by a compressed gas system. In the system, the pressure applied to the limb through the cuff is pre-determined to maintain adequate pressure to control bleeding while preventing unnecessary tissue damage. The pressure is also pre-set prior to inflation of the cuff.

In a preferred embodiment, the interior of the cuff is hollow with an air-tight, void. The void is connected to the exterior by a valve system that permits pressurized gas to be admitted to the void. In the preferred embodiment, air, or other non-flammable gas, such as CO₂, is inserted into the void under pressure via a gas cylinder or canister, wherein the system monitors and pressurizes the void to a pre-determined pressure. In the preferred embodiment, the inflation pressure is predetermined to ensure optimal blood loss, while minimizing tissue damage due to over-pressurization of the tourniquet.

In another embodiment the pressure of gas is determined by the cylinder or canister comprising a pre-determined gas pressure, such that when the gas is released into the void, optimal pressure is achieved when the pressure in the gas cylinder and void has reached an equilibrium pressure. In another embodiment, the valve on the cuff has a pressure cut-off, such that upon reaching a certain pressure, no further gas is permitted into the void. The valve, in this case is one-way, such that gas can enter the void but not escape. In another embodiment, a cylinder holder contains a valve system that regulates the pressure permitted into the void. In another embodiment, the pressure can be adjusted prior to use.

### BRIEF DESCRIPTION OF DRAWINGS

FIG. 1. Cross-sectional diagram of the tourniquet illustrating the wrap-around application of the cuff, attachment means, such as hook and loop fasteners, the internal bladder and valve placement. An alternative embodiment comprises a hollow tube with an internal hollow space to insert a limb. In this configuration, no attachment means is needed. The wearer merely slips their limb into the hollow space.

FIG. 2. Illustration of gas dispenser showing some of the components of the gas dispenser. Illustrated is the gas cylinder, valve stem, connection port, trigger mechanism, and dispenser valve arrangement.

### DETAILED DESCRIPTION OF PREFERRED EMBODIMENTS

A rapid, easy to apply application of a tourniquet is essential to control, especially under circumstances removed from trained emergency medical assistance. Application of a means to control bleed through a tourniquet requires adequate pressure that is at the same time not too great to cause tissue damage. Over-pressure of a tourniquet can cause injury or even loss of the limb it is placed on.

Application of adequate pressure is often difficult under “field settings”, especially if the injured individual is alone or accompanied by non-medically training individuals. Because application of adequate pressure can cause significant discomfort, applying a tourniquet alone or without trained assistance can lead to applying an inadequate pressure to stem bleeding. Application of inadequate pressure is even more of a danger in circumstances were the injured individual is alone and in a weakened condition.

### Example 1

### Rapid Tourniquet System Primary Components

An important functional aspect of the tourniquet system is a device wherein an individual, whether medically trained or not, can effectively insert the tourniquet on himself/herself or on another person to effectively control bleeding, while minimizing tissue damage. Therefore, an important aspect is a device that enables application of a pre-determined and pre-set pressure so that an individual applying the tourniquet can apply the tourniquet rapidly with the correct pressure to the injured limb. The device is applicable to medical treatment facility use or for use in remote areas or even under field conditions.

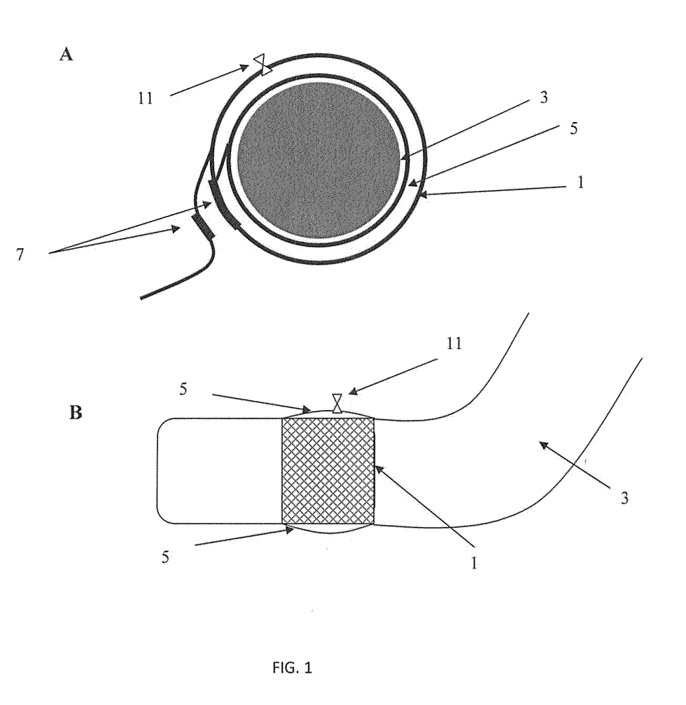

An example of a preferred embodiment is illustrated in FIG. 1. The device comprises a tourniquet cuff (1), made of an air-tight material containing an inner, outer surface and an internal void or bladder (5) that is gas-tight. When in use, the cuff (1) is secured around the limb (3) such that the inner surface of the cuff (1) is next to or closest to the limb (3).

The cuff (1) can be constructed in variable dimensions and made of any number of materials, including para-aramid synthetic fiber (e.g., Kevlar) or other durable, flexible material. For example, in a preferred embodiment, the cuff can be 2 to 10 inches long to accommodate individuals or limbs of specific sizes or able to be adjusted to accommodate a wide range of sizes. After the cuff (1) is applied around the limb by wrapping the cuff around the limb, it can be firmly fitted on the limb via a quick application attachment, such as a loop and hood connection (7), such as VELCRO™. Alternatively, the cuff (1) can be constructed as hollow tube, preferably made of an elastic material. In this embodiment, the cuff (1) comprises an air-tight hollow tube comprising an internal diameter to insert a limb and a said gas-tight internal void (5) encompassing the walls of the internal diameter and running the entire length of the hollow tube. In this embodiment, the material is made of a flexible material to enable easy and firm fitting over the limb. The wearer would merely slip the limb into the cuff (1). In this alternative, no fastening would be necessary. In one embodiment, multiple sizes would be available to ensure a firm fit prior to inserting gas into the internal void (5).

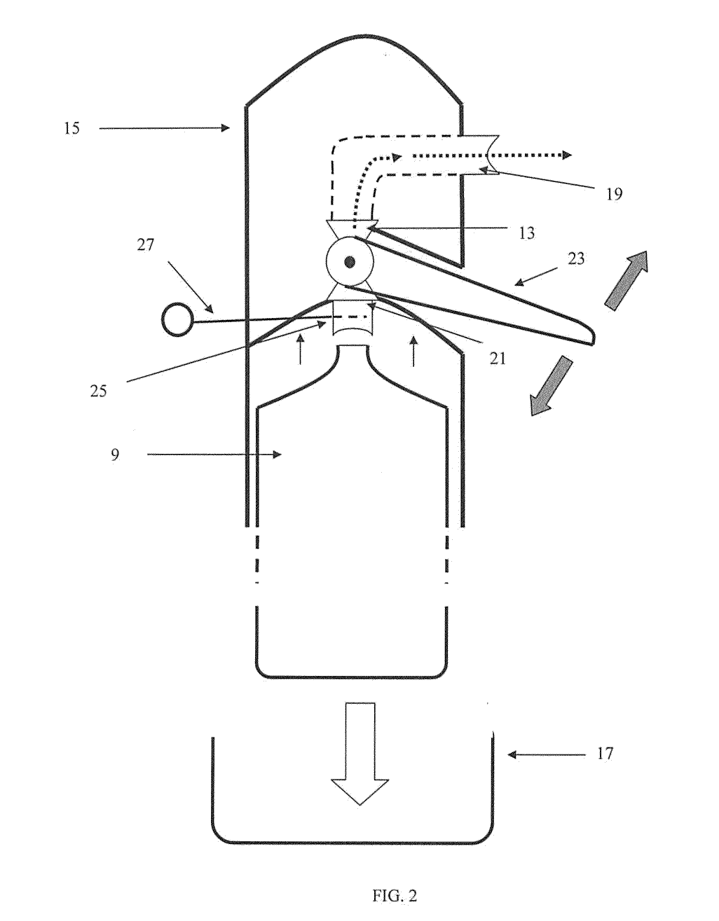

Pressure, to stem bleeding of the limb, is applied to the tissue to control bleeding by the cuff (1) but administration of gas, such as CO₂, into the bladder from a small cylinder (9) (FIG. 2) via a valve (11) connected to the cuff (1) that protrudes into the bladder (5). In this embodiment, the gas cylinder (9) (FIG. 2) is connected to the valve on the cuff (11) (FIG. 1). Gas is dispensed into the cuff at a pre-set pressure that is pre-determined to be appropriate to prevent bleeding while not causing further tissue damage. In other embodiments, the gas cylinder (9) is housed in a gas dispenser (15).

The gas pressure in the void (5) is determined in a number of alternative means. In one alternative, the gas cylinder (9) comprises gas at a pre-determined pressure. When the gas is released into the void, optimal pressure is achieved when the pressure in the gas cylinder and void has reached an equilibrium pressure. In another alternative embodiment, the valve on the cuff comprises a pressure cut-off. In another embodiment, a gas dispenser (15) contains a valve system that regulates the pressure permitted into the void. In these alternatives, pressure settings of the valves can be preset or, alternatively, the valves can comprise an adjustment to enable setting of the pressure cut-off. An one embodiment, the valve is a pop-off valve or relief valve that releases gas at a pre-set pressure or pressure set immediately prior to administering tourniquet. In this embodiment, gas enters the void (bladder). When the pre-set pressure is attained, the value does not permit further gas to enter.

In another embodiment, a gas dispenser (15), containing a gas cylinder (9) is integral to the bladder. In one embodiment, no attachment of the dispenser to the valve (11) is necessary since the connecting port (19), where gas can leave the gas cylinder (9), on the gas dispenser (15) (FIG. 2) is pre-connected to the value (11) FIG. 1. In another embodiment, connection of the connecting port (19) is made to the valve (11) when ready to inflate the cuff. The user merely inserts the cuff (1) over the limb attaches the gas dispenser (15) via the connecting port (19) to the valve (11) and activates the gas dispenser (15) via a button or trigger mechanism (23) on the gas dispenser. Gas enters, as above, at a pre-set pressure, pre-determined appropriate to prevent bleeding without causing undue tissue damage. The pre-set pressure being obtained either when equilibrium between the gas cylinder and cuff are achieved by or by other means such as a pop-of or relief valve in the gas dispenser (13) (see Example 2) or the valve on the cuff (11).

Tourniquet pressure, determined by the compressed gas cylinder or at the valve is that level considered adequate to control bleeding, without incurring tissue damage by overpressure of the cuff (1). Typically, this is set within the range between 180 to 220 mmHg, although other pressure settings are possible depending on factors, such as limb applied and whether worn over or under garments. Typically, the preset pressure can be 200 to 220 mmHg.

Pre-set pressure in the cuff can be obtained by the pressure of gas in the gas cylinder (9). In this embodiment, pressure in the cuff is obtained once equilibrium between the pressure in the cuff and the cylinder is reached. As an alternative to proper pressure being maintained by the pre-set pressure in the gas cylinder, proper pressure can be maintained by the valve (11) contains a means that or by a valve in a gas dispenser (15). As an example, which is illustrated in FIG. 1, the valve (11) only permits a preset pressure of gas in the bladder. In this example, connection of a gas cylinder to the valve (11) causes the void (5) to fill with gas to inflate the cuff (1). The valve will only permit gas to enter but not escape upon disconnection of the cylinder from the valve, unless desired by pressing of a gas release button or switch on the valve (11).

### Example 2

### Gas Dispenser System for Housing Gas Cylinder

In an alternative system, the gas cylinder and potentially the pressure regulation means is incorporated into a gas dispenser (15), which comprises the cylinder (9) housed and valve system (13). This is illustrated as an example in FIG. 2. In one embodiment, gas pressure is regulated by the valve (13). As an example, a valve (13) is connected to the cylinder (9), as illustrated in FIG. 2.

Whether the gas dispenser is used or as described in Example 1, an important aspect is a tourniquet system where pressure can be applied to a limb relatively rapidly and easily at a pre-determine/pre-set pressure. In one embodiment, the device is pre-set so that no further adjustment of pressure is needed by the user prior to inflation of the cuff. As an example, gas pressure in the cylinder is pre-set with the correct pressure. In this embodiment, the pressure in the cuff void (5), upon attaching the cylinder to the valve on the cuff (11) and opening the cylinder, the proper inflation of the cuff is achieved in seconds. Similarly, in another embodiment, the valve (11) on the cuff (1) or the valve (13) in the gas dispenser (15) is pre-set to an appropriate pressure. These features permits even untrained individuals to rapidly apply the tourniquet properly. The gas dispenser (FIG. 2) enables even easier handling of the cylinder (9) and dispensing of gas.

In another embodiment, the pressure allowed into the bladder (5) can be adjusted on the dispenser valve (13) (FIG. 2) or at the cuff valve (11). As mentioned, in a preferred embodiment, the valves are one-way valves, such that gas can enter the void but not escape unless permitted to escape by pressing a gas release button or switch on the valve. The valve (11) on the cuff (1) can be used to release gas from the cuff (1) in order to deflate it for its removal or re-use.

As mentioned above, for greater ease of use, the gas cylinder (9) fits into a gas dispenser (15), comprising a cylinder case (17), a connecting port (19) to the valve (11). The connecting port can be any system or means for connecting to the valve such as a quick-connect fitting used to connect pipes. An example of this embodiment is illustrated in FIG. 2. In this example, the cylinder (9) fits into the cylinder holder case (17) of the gas dispenser (15). The gas dispenser (15), then fits over and connects to the cylinder case (17). The cylinder case (17) provides protection to the gas cylinder preventing damage to the cylinder.

On inserting the gas cylinder (9) into the cylinder gas dispenser (15), the top of the cylinder (9) is inserted into the valve stem (21). The valve stem (21) is connected to the dispenser valve (13). The dispenser valve (13) can comprise a simple valve that permits the flow of gas to the connecting port (19) or can contain a regulator that is pre-set such that it automatically enables the flow of gas to a pre-determined pressure in the internal void of the cuff upon initiation of gas flow. Alternatively, the regulator can contain an adjustment means to vary the pressure of gas flow to the internal void (5) of the cuff (1).

In the above example, the cylinder (9) is inserted into the cylinder holder (17) until the top of the cylinder (9) inserts into the valve stem (21). The valve stem (21) serves as an attachment means or port connecting the cylinder (9) via the dispenser valve (13). Gas can be released by opening the dispenser valve (13) by application of a gas release mechanism. Although any gas release mechanism is possible, an illustrative example is a trigger-like feature (21). Alternative gas release mechanisms can be a switch or button. The trigger feature enables easier inflation of gas to the cuff (1), which is especially important when applying the tourniquet alone, where the wearer may be in a partially weakened condition.

In one embodiment, the dispenser valve (13) contains a pressure monitoring device that is either pre-set or that is adjustable to variable pressure settings. In another embodiment, the valve stem (21) also contains a pin or activating mechanism (25), whereby the cylinder, which is normally sealed, can be activated or open to the valve stem (21) and dispenser valve (13) by pulling of a pin (27) or other means.

### Example 3

### Method of Controlling Bleeding From a Limb

The device of example 1 and 2 can be utilized to rapidly control bleeding from a limb, even if the injured individual is not medically trained. The method comprises the steps: applying the tourniquet cuff of claim 1; inflating said cuff using compressed gas using the system of claim 1 until the pressure in the internal void of the cuff is at the pre-determined pressure. In this method a pre-determined pressure is pre-set within the range of 180 to 220 mmHg. The pre-set pressure is selected to give adequate pressure to the injured limb, effectively controlling bleeding while minimizing tissue damage. In this method, either of the alternative cuff (1) arrangements can be utilized, as discussed in Example 1 (FIG. 1).

The inventive method can also utilize gas dispenser (15) as in FIG. 2. In this embodiment, the cuff (1) is applied to the injured limb. The gas dispenser (15) is connected to the valve on the cuff (1) via a connecting means such as a connecting port (19). The connecting port can be any system such as a quick-connect fitting used to connect pipes. Premature release of gas is prevented through the use of an activating mechanism (25). This can pin that upon pulling enables gas to be dispensed from the gas cylinder. The flow of gas is then initiated by activating the flow of gas by manipulating the lever, button or switch (23) on the gas dispenser (15) to enable gas to flow into the cuff. If the dispenser valve or valve on the cuff comprises an adjustment to pre-set the gas pressure allowed to flow, the pressure is set prior to activating the flow of gas into the cuff.

Obviously, many modifications and variations of the present invention are possible in light of the above teachings. It is therefore to be understood that, within the scope of the appended claims, the invention may be practiced otherwise than as specifically described.

What is claimed is:

1. A tourniquet system for controlling hemorrhage from a limb, comprising a cuff comprising an air-tight material containing a gas-tight internal void connected to a gas cylinder wherein gas from said cylinder is capable of entering and filling said gas-tight internal void of said cuff at a pre-determined or pre-set pressure.

2. The tourniquet system of claim 1, wherein said cuff contains a means for firmly attaching the cuff around the limb and a valve connecting the outside of the cuff to the gas-tight internal void.

3. The tourniquet system of claim 1, wherein the gas cylinder contains gas at a pre-set or pre-determined pressure appropriate for proper inflation of the cuff.

4. The tourniquet system of clam 1, wherein said cuff is 2 to 10 inches long.

5. The tourniquet system of claim 1, wherein said gas cylinder is enclosed by a gas dispenser comprising: a valve stem connecting the gas cylinder head to a dispenser valve, which is connected to a connecting port, wherein said connecting port is a means for connecting the gas dispenser to said valve on said cuff connecting the outside of the cuff to the gas-tight internal void, wherein gas can flow from the gas cylinder through the connecting port to said cuff, and a means for initiating flow of gas from the cylinder through the dispenser valve ultimately to the gas-tight internal void.

6. The tourniquet system of claim 1, wherein the predetermined pressure is set within the range 180 to 220 mmHg.

7. The tourniquet system of claim 1, wherein said cuff is an air-tight hollow tube comprising an internal diameter to insert a limb and said gas-tight internal void encompassing the walls of the internal diameter and running the entire length of the hollow tube.

8. The tourniquet system of claim 1, wherein said cuff can be fit around a limb and secured via a fastening means.

9. The tourniquet system of claim 2, wherein said valve or a valve connected to the gas cylinder comprises a means for maintaining the gas pressure entering the gas-tight internal void at said pre-determined or pre-set pressure.

10. The tourniquet system of claim 2, wherein the valve is a one-way valve that only permits gas to enter the internal void but can permit gas to exit by pushing gas release switch or button.

11. The tourniquet system of claim 2, wherein said means for regulating the gas entering the gas-tight internal void automatically at a predetermined pressure comprises a further means, wherein the predetermined pressure can be adjusted.

12. The tourniquet system of claim 6, wherein said gas dispenser comprises a gas regulator that regulates the gas pressure entering the gas-tight internal void automatically at a predetermined pressure.

13. The tourniquet system of claim 6, wherein said means for initiating flow of gas is an external trigger mechanism, switch, or button connected to the dispenser to a dispenser valve that is connected to the valve stem of the gas cylinder so that upon manipulation of the trigger, switch or button the dispenser valve is opened and gas is permitted to flow out of the gas cylinder through the valve stem and dispenser valve to the valve connection between the outside of the cuff to the gas-tight internal void on the cuff.

14. The tourniquet system of claim 6, wherein said valve stem contains an deactivating means to disable and prevent gas from leaving the gas cylinder through the valve stem.

15. The tourniquet system of claim 11, wherein said fastening means is a loop and hook connection.

16. A method of controlling bleeding from a limb, comprising the steps: applying the tourniquet cuff of claim 1; inflating said cuff using compressed gas using the system of claim 1 until the pressure in the internal void of the cuff is at the pre-determined pressure.

17. The method of claim 16, wherein said inflation of said cuff is by a pre-set pressure in the range of 180 to 220 mmHg.

18. The method of claim 16, further comprising the step of determining the gas pressure to be inserted in the internal void and adjusting the pressure setting of the gas.

19. The method of claim 16, wherein said gas cylinder of claim 1 is enclosed in a gas dispenser of claim 6.

20. The method of claim 19, wherein said inflating step is initiated by application of a gas release mechanism wherein said gas release mechanism is an external trigger mechanism, switch or button connected to the dispenser valve enabling gas to flow from the gas cylinder through said connecting port and valve connecting the outside of the cuff to the gas-tight internal void.
